(19) United States
(12) Patent Application Publication
(10) Pub. No.: US 2024/0234738 A9
(43) Pub. Date: Jul. 11, 2024

(54) SAMPLES INCLUDING LITHIUM OR NON-REACTIVE LITHIUM MIMICS FOR NONDESTRUCTIVE TESTING TECHNOLOGIES IN AMBIENT CONDITIONS

(71) Applicant: GM GLOBAL TECHNOLOGY OPERATIONS LLC, Detroit, MI (US)

(72) Inventors: Robin JAMES, Rochester Hills, MI (US); Megan E. MCGOVERN, Detroit, MI (US); Dmitriy BRUDER, Clinton Twp., MI (US); Whitney Ann POLING, Rochester Hills, MI (US); Erik Damon HUEMILLER, Troy, MI (US); Shaomao XU, Sterling Heights, MI (US); Caleb REESE, Ferndale, MI (US)

(73) Assignee: GM GLOBAL TECHNOLOGY OPERATIONS LLC, Detroit, MI (US)

(21) Appl. No.: 17/972,063

(22) Filed: Oct. 24, 2022

(51) Int. Cl. H01M 4/62; H01M 4/04; H01M 4/66

(52) U.S. Cl. CPC H01M 4/626; H01M 4/0404; H01M 4/661

(57) ABSTRACT

The present disclosure provides electrode assemblies for safe nondestructive testing in ambient conditions. In certain variations, the electrode assembly includes a current collector and a non-reactive lithium mimic material disposed on or near one or more surfaces of the current collector or embedded in the current collector. In other variations, the electrode assembly includes include a current collector, a lithium metal material disposed on or near one or more surfaces of the current collector or embedded in the current collector to form an electrode assembly, and a non-conductive pouch holding the electrode assembly.

This section provides background information related to the present disclosure which is not necessarily prior art.

Advanced energy storage devices and systems are in demand to satisfy energy and/or power requirements for a variety of products, including automotive products such as start-stop systems (e.g., 12V start-stop systems), battery-assisted systems, hybrid electric vehicles (“HEVs”), and electric vehicles (“EVs”). Typical lithium-ion batteries include at least two electrodes and an electrolyte and/or separator. One of the two electrodes may serve as a positive electrode (or cathode) and the other electrode may serve as a negative electrode (or anode). A separator filled with a liquid or solid electrolyte may be disposed between the negative and positive electrodes. The electrolyte is suitable for conducting lithium ions between the electrodes and, like the two electrodes, may be in solid and/or liquid form and/or a hybrid thereof. In instances of solid-state batteries, which include solid-state electrodes and a solid-state electrolyte (or solid-state separator), the solid-state electrolyte (or solid-state separator) may physically separate the electrodes so that a distinct separator is not required.

Many different materials may be used to create components for a lithium-ion battery. For example, the negative electrode may be defined by a lithium-containing material, such as metallic lithium or lithium-containing alloys, so that the electrochemical cell is considered a lithium metal battery or cell. Metallic lithium for use in the negative electrode of a rechargeable battery has various potential advantages, including having the highest theoretical capacity and lowest electrochemical potential. Thus, lithium metal batteries are one of the most promising candidates for high energy storage systems.

One issue that arises is the high level of reactivity of the lithium metal, which can result in interfacial instability and undesired reactions with various species when the lithium metal is exposed during manufacture, testing, and/or operation of the electrochemical cell (e.g., potentially leading to dendrite formation). As such, no in-line quality verification method currently exists for electrochemical cells incorporating lithium metal anodes. The application of nondestructive testing (NDT) technologies to lithium metal anodes is often challenging at least because lithium metal is not stable at standard atmospheric conditions, as it readily reacts with oxidizing species like water vapor from environmental humidity to form a strong base, lithium hydroxide and hydrogen gas. Because of this potentially exothermic reaction, lithium metal anodes are only be handled in a glovebox (e.g., less than about 10 ppm water), dry room (e.g., less than about 20% relative humidity), or with one or more protective material coatings. Accordingly, it would be desirable to develop improved materials and methods for developing and evaluating nondestructive evaluation (NDE) tests and methods to be used for nondestructive, quality testing of lithium-containing anodes, like lithium metal anodes.

### SUMMARY

This section provides a general summary of the disclosure, and is not a comprehensive disclosure of its full scope or all of its features.

The present disclosure relates to materials and methods for nondestructive, quality testing and more particularly to samples including lithium or non-reactive lithium mimics for nondestructive, quality testing.

In various aspects, the present disclosure provides an electrode assembly for nondestructive testing in ambient conditions. The electrode assembly may include a current collector and a non-reactive lithium mimic material disposed on or near one or more surfaces of the current collector or embedded in the current collector.

In one aspect, the non-reactive lithium mimic material may have an electrical conductivity that is within about 10% of an electrical conductivity of lithium.

In one aspect, the non-reactive lithium mimic material may have an electrical conductivity greater than or equal to about 7×10⁶ S/m to less than or equal to about 15×10⁶ S/m.

In one aspect, the non-reactive lithium mimic material may have a thermal conductivity that is within about 10% of a thermal conductivity of lithium.

In one aspect, the non-reactive lithium mimic material may have a thermal conductivity greater than or equal to about 60 W/(m·K) to less than or equal to about 100 W/(m·K).

In one aspect, the non-reactive lithium mimic material may be selected from the group consisting of: indium, tin, iron, nickel, chromium, and alloys thereof.

In one aspect, the non-reactive lithium mimic material may define a continuous layer having one or more first regions with a first thickness and one or more second regions with a second thickness, where the first thickness is different from the second thickness.

In one aspect, the non-reactive lithium mimic material may be disposed on a first surface of the current collector and an interface defined between the non-reactive lithium mimic material and the current collector may include one or more selected regions of delamination.

In one aspect, the non-reactive lithium mimic material may define a discontinuous layer over the one or more surface of the current collector.

In one aspect, the electrode assembly may further include a continuous electroactive material layer having a major dimension parallel with a major dimension of the current collector, where the non-reactive lithium mimic material overlaps a portion of the non-reactive lithium mimic and a portion of the electroactive material layer at an interface of the electroactive material layer and the current collector.

In one aspect, the non-reactive lithium mimic material may be disposed on or near the one or more surfaces of the current collector and the electrode assembly may be sealed in a non-conductive pouch.

In one aspect, the non-reactive lithium mimic material may be embedded in the current collector, and the current collector may include a copper mesh or a copper expanded metal foil.

In various aspects, the present disclosure provides an assembly for nondestructive testing in ambient conditions. The electrode assembly may include a current collector, a lithium metal material or non-reactive lithium mimic material disposed on or near one or more surfaces of the current collector or embedded in the current collector to form an electrode assembly, and a non-conductive pouch holding the electrode assembly.

In one aspect, the lithium metal material or non-reactive lithium mimic material defines a continuous layer having one or more first regions with a first thickness and one or more second regions with a second thickness, where the first thickness is different from the second thickness.

In one aspect, the lithium metal material or non-reactive lithium mimic material may define a discontinuous layer over the one or more surfaces of the current collector.

In one aspect, the lithium metal material or non-reactive lithium mimic material may be embedded in the current collector, and the current collector may include a copper mesh or a copper expanded metal foil.

In one aspect, the electrode assembly may further include a continuous electroactive material layer having a major dimension parallel with a major dimension of the current collector, where the non-reactive lithium mimic material overlaps a portion of the non-reactive lithium mimic and a portion of the electroactive material layer at an interface of the electroactive material layer and the current collector.

In one aspect, the non-reactive lithium mimic material may have an electrical conductivity greater than or equal to about 7×10⁶ S/m to less than or equal to about 15×10⁶ S/m.

In one aspect, the non-reactive lithium mimic material may have a thermal conductivity greater than or equal to about 60 W/(m·K) to less than or equal to about 100 W/(m·K).

In one aspect, the non-reactive lithium mimic material may be selected from the group consisting of: indium, tin, iron, nickel, chromium, and alloys thereof.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is an illustration of an example electrochemical battery cell including a non-reactive lithium mimic in accordance with various aspects of the present disclosure;

FIG. 2 is an illustration of an example electrode assembly including a non-reactive lithium mimic disposed on or near a current collector in accordance with various aspects of the present disclosure;

FIG. 3 is an illustration of an example electrochemical battery cell including a lithium-containing material surrounded by a pouch material in accordance with various aspects of the present disclosure;

FIG. 4 is an illustration of an example electrode assembly including a lithium-containing material disposed on or near a current collector and surround by a pouch material in accordance with various aspects of the present disclosure; and

FIG. 5 is an illustration of an example electrode assembly including a lithium-containing material embedded in a copper substrate in accordance with various aspects of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected, or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates both exactly or precisely the stated numerical value, and also, that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

After manufacturing a product such as a battery cell, testing may be performed to ensure that the product is manufactured correctly. The manufacturer, or a third party, develops nondestructive tests to validate various characteristics of the product or to detect defects. That way, some or all of the products can be tested after manufacture, and prior to sale, to improve quality and/or to ensure consistent performance When developing testing for a product, a manufacturer typically creates mock-up samples using the same materials as the production product. However, battery cells include certain reactive materials, like lithium metal. Therefore, it can be difficult to determine the effectiveness of various nondestructive testing technologies on the battery cells since battery mock-up samples including lithium metal are not stable outside of a glovebox/dry room and pose difficulties in being sent directly to testing companies for development purposes.

The present disclosure relates to materials and methods for nondestructive, quality testing and more particularly to samples including lithium or non-reactive lithium mimics for nondestructive, quality testing. As discussed above, lithium-containing negative electroactive materials, such as lithium metal, may be highly reactive, especially with oxidizing species. In certain aspects, an electrode assembly may include a non-reactive lithium mimic material. For example, in various aspects, a non-reactive lithium mimic material has material properties like (or that replicate the material properties of) lithium metal (and/or lithium metal anodes in electrochemical cells) may be used to enable evaluation of the suitability of nondestructive testing technologies, for example, outside of a glove box or dry room at normal atmospheric conditions. In certain aspects, battery mock-up samples may include a portion (e.g., two or more active components) of a battery cell and at least one layer including a non-reactive lithium mimic material, which may substitute for or otherwise represent a lithium-containing material. In certain aspects, the battery mock-up samples can be handled without special handling precautions, for example, in atmospheric conditions outside of a glovebox. The lithium metal battery mock-up samples may be used to evaluate distinct types of nondestructive testing technologies and their respective abilities to identify characteristics and/or defects of the lithium metal layer.

In certain variations, the non-reactive lithium mimic materials may have similar physical, acoustical, atomic, chemical, electrical, magnetic, manufacturing, mechanical, optical, thermal, and/or radiological material properties as lithium metal or a lithium-containing alloy (or of a lithium metal anode in an electrochemical cell). In other variations, the non-reactive lithium mimic material may replicate certain material properties, like the physical, acoustical, atomic, chemical, electrical, magnetic, manufacturing, mechanical, optical, thermal, and/or radiological material properties, of lithium metal or a lithium-containing alloy (or of a lithium metal anode in an electrochemical cell). For example, in certain variations, the non-reactive lithium mimic material may include nonreactive metals, like indium, tin, iron, chromium, nickel, and alloys and combinations thereof, that mimic the properties to be measured by the technology being evaluated. By non-reactive, it is meant that the material undergoes minimal adverse or undesired chemical reactions with other chemicals in a surrounding environment, especially having reduced or diminished reactivity with oxidizing species, including water. Thus, the non-reactive lithium mimic material may improve the safety in handling (including shipping) samples for nondestructive evaluation (NDE) testing.

The non-reactive lithium mimic material may thus have one or more properties that are quite similar to lithium (Li) and thus may serve as a material that mimics lithium for purposes of nondestructive evaluation (NDE) testing. In certain variations, the non-reactive lithium mimic material may have an electrical conductivity that is within about 10%, optionally within about 8%, optionally within about 5%, and in certain variations, optionally within about 2%, of an electrical conductivity of lithium and/or a thermal conductivity that is within about 10%, optionally within about 8%, optionally within about 5%, and in certain variations, optionally within about 2%, of the thermal conductivity of lithium. For example, the non-reactive lithium mimic material may have an electrical conductivity greater than or equal to about 9×10⁶ S/cm to less than or equal to about 11×10⁶ S/cm, and the non-reactive lithium mimic material may have a thermal conductivity greater than or equal to about 76.5 W/(m·K) to less than or equal to about 93.5 W/(m·K). In other variations, the non-reactive lithium mimic material may have an electrical conductivity greater than or equal to about 7×10⁶ S/m to less than or equal to about 15×10⁶ S/m, and the non-reactive lithium mimic material may have a thermal conductivity greater than or equal to about 60 W/(m·K) to less than or equal to about 100 W/(m·K).

Indium (In) may mimic the electrical conductivity, thermal conductivity, melting point, hardness, tensile strength, and other mechanical properties (e.g., stiffness, density) of lithium, such that indium can exhibit similar behavior as lithium during processing (including, for example, using common rolling or extrusion processes) and/or during mechanical testing. Generally, indium may have an elastic modulus (e.g., Young's modulus (E)) of about 12.7 GPa, while lithium has an elastic modulus (e.g., Young's modulus (E)) of about 9.5 GPa; indium may have a shear modulus of about 3.8 GPa, while lithium has a shear modulus of about 4.25 GPa; indium may have a Poisson's ratio of about 0.45, while lithium has a Poisson's ratio of about 0.362; and indium may have a yield strength of about 0.93 MPa, while lithium has a yield strength of about 0.362 MPa. Further, indium may have a homologous temperature close to lithium, where homologous temperature plays a significant role in the creep behavior of the respective materials. For example, the homologous temperature of indium may be about 0.69, while the homologous temperature of lithium is about 0.66, at room temperature (e.g., greater than or equal to about 20° C. to less than or equal to about 25° C., and in certain aspects, optionally about 24.4° C.). Further still, indium may have an electrical conductivity that is about 12×10⁶ S/m, while lithium has an electrical conductivity of about 11×10⁶ S/m, such that the respective materials have similar behaviors in the instance of nondestructive evaluation (NDE) tests based on electrical or thermal conductivity.

Further, tin (Sn) and nickel (Ni) may have similar electrical and thermal conductivities to lithium and may thus likewise serve as a lithium mimic material. Further, iron (Fe) and chromium (Cr) may have the same crystal structure as lithium and similar electrical and thermal conductivities to lithium. Further still, iron has a similar electrical conductivity to lithium—for example, about 10×10⁶ S/m for iron versus about 11×10⁶ S/m for lithium). In most instances, in accordance with certain aspects of the present disclosure, it is more important to mimic the differences in material properties rather that absolute values for a lithium mimic material. For example, the electrical conductivity difference between lithium and copper is about 49×10⁶ S/m. Many combinations of conductive materials that mimic this difference can be used to validate electromagnetic nondestructive measurements which rely on this difference, such as eddy current testing.

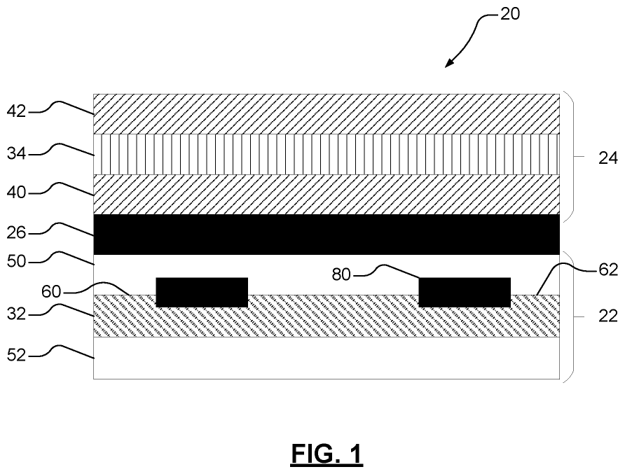

The non-reactive lithium mimic material may thus be used to form samples for nondestructive testing of certain properties (e.g., defects arising) during manufacturing of lithium metal and/or during the operation of electrochemical cells including lithium metal anodes. For example, in certain variations, the non-reactive lithium mimic material may be used to characterize, verify, or identity thicknesses, cracks and the like during in-line roll-to-roll processing. The non-reactive lithium mimic may also be used to characterize, verify, or identify certain contamination of the lithium metal (or lithium metal anode). Further still, the non-reactive lithium mimic may be used to characterize, verify, or identify lithium plating in electrochemical cells (see, e.g., FIG. 1). Nondestructive testing techniques can include without limitation, electromagnetic techniques or methods such as eddy current, acoustic techniques or methods such as ultrasound or phased array, infrared techniques or methods such as thermography, and/or x-ray techniques or methods such as radiography.

In various aspects, as illustrated in FIG. 1, a non-reactive lithium mimic material 80 may be disposed between layers of a representative electrochemical cell 20 to replicate lithium plating as developed during cycling. The electrochemical cell 20 may be a dry cell with no voltage generation that includes a first (or positive) electrode 24 physically separated from a second (or negative) electrode 22 by a separating layer 26. Although a single positive electrode 24 and a single negative electrode 22 are illustrated, the skilled artisan will recognize that the present teachings also extend to various other configurations, including those having additional layers (e.g., one or more positive electrodes (or cathodes) and one or more negative electrodes (or anodes), as well as various other current collectors with one or more electroactive material layers disposes on one or more surfaces thereof) or fewer layers (e.g., electroactive assembly including the non-reactive lithium mimic and a current collector).

As illustrated, the positive electrode 24 may include a first (or positive) current collector 34 and first and second positive electroactive material layers 40, 42 disposed on opposing sides of the positive current collector 34. The positive current collector 34 may be a metal foil, metal grid or screen, or expanded metal. The positive current collector 34 may comprise aluminum (Al) or any other appropriate electrical conductive material known to those of skill in the art.

The first positive electroactive material layer 40 may be the same as or different from the second positive electroactive material layer 42. The first and second positive electroactive material layers 40, 42 may include positive electroactive materials, for example, lithium-based active materials, that are capable of undergoing lithium intercalation and deintercalation, alloying and dealloying, or plating and stripping, while functioning as the positive terminal of a lithium-ion battery. The positive electroactive material layers 40, 42 can be defined by a plurality of electroactive material particles (not shown). Such positive electroactive material particles may be disposed in one or more layers so as to define the three-dimensional structure of the positive electroactive material layers 40, 42.

In various aspects, the positive electroactive materials include layered oxides represented by LiMeO₂, where Me is a transition metal, such as cobalt (Co), nickel (Ni), manganese (Mn), iron (Fe), aluminum (Al), vanadium (V), or combinations thereof. In other variations, the positive electroactive materials include olivine-type oxides represented by LiMePO₄, where Me is a transition metal, such as cobalt (Co), nickel (Ni), manganese (Mn), iron (Fe), aluminum (Al), vanadium (V), or combinations thereof. In still other variations, the positive electroactive materials include monoclinic-type oxide represented by Li₃Me₂(PO₄)₃, where Me is a transition metal, such as cobalt (Co), nickel (Ni), manganese (Mn), iron (Fe), aluminum (Al), vanadium (V), or combinations thereof. In still other variations, the positive electroactive materials include spinel-type oxides represented by LiMe₂O₄, where Me is a transition metal, such as cobalt (Co), nickel (Ni), manganese (Mn), iron (Fe), aluminum (Al), vanadium (V), or combinations thereof. In still other variations, the positive electroactive materials can include LiMeSO₄F and/or LiMePO₄F, where Me is a transition metal, such as cobalt (Co), nickel (Ni), manganese (Mn), iron (Fe), aluminum (Al), vanadium (V), or combinations thereof. In still further variations, the positive electroactive material layers 40, 42 may be composite layers including a combination of positive electroactive materials. For example, one or both of the first and second positive electroactive material layers 40, 42 may include a first positive electroactive material and a second electroactive material. A ratio of the first positive electroactive material to the second positive electroactive material may be greater than or equal to about 5:95 to less than or equal to about 95:5. In certain variations, the first and second positive electroactive materials may be independently selected from one or more layered oxides, one or more olivine-type oxides, one or more monoclinic-type oxides, one or more spinel-type oxide, LiMeSO₄F, LiMePO₄F, or combinations thereof, where Me is a transition metal, such as cobalt (Co), nickel (Ni), manganese (Mn), iron (Fe), aluminum (Al), vanadium (V), or combinations thereof.

In each variation, the positive electroactive materials may be optionally intermingled with an electronically conductive material (i.e., conductive additive) that provide an electron conductive path and/or a polymeric binder material that improve the structural integrity of the positive electrode 24. For example, each positive electroactive material layer 40, 42 may include greater than or equal to about 70 wt. % to less than or equal to about 98 wt. %, and in certain aspects, optionally greater than or equal to about 80 wt. % to less than or equal to about 97 wt. %, of the positive electroactive material; greater than or equal to 0 wt. % to less than or equal to about 30 wt. %, and in certain aspects, optionally greater than or equal to about 0.5 wt. % to less than or equal to about 10 wt. %, of the electrical conducting material; and greater than or equal to 0 wt. % to less than or equal to about 20 wt. %, and in certain aspects, optionally greater than or equal to about 0.5 wt. % to less than or equal to about 10 wt. %, of the polymeric binder.

Example polymeric binders include polyimide, polyamic acid, polyamide, polysulfone, polyvinylidene difluoride (PVdF), polytetrafluoroethylene (PTFE), polyacrylic acid (PAA), blends of polyvinylidene fluoride and polyhexafluoropropene, polychlorotrifluoroethylene, ethylene propylene diene monomer (EPDM) rubber, carboxymethyl cellulose (CMC), nitrile butadiene rubber (NBR), styrene-butadiene rubber (SBR), lithium polyacrylate (LiPAA), sodium polyacrylate (NaPAA), sodium alginate, and/or lithium alginate. Electronically conducting materials may include, for example, carbon-based materials, powdered nickel or other metal particles, or conductive polymers. Carbon-based materials may include, for example, particles of graphite, acetylene black (such as KETCHEN™ black or DENKA™ black), carbon nanofibers and nanotubes (e.g., single wall carbon nanotubes (SWCNT), multiwall carbon nanotubes (MWCNT)), graphene (e.g., graphene platelets (GNP), oxidized graphene platelets), conductive carbon blacks (such as, SuperP (SP)), and the like. Examples of a conductive polymer include polyaniline, polythiophene, polyacetylene, polypyrrole, and the like.

The separating layer 26 may be a porous separator having a porosity greater than or equal to about 30 vol. % to less than or equal to about 80 vol. %. For example, in certain instances, the separator 26 may be a microporous polymeric separator that includes a polyolefin. The polyolefin may be a homopolymer (derived from a single monomer constituent) or a heteropolymer (derived from more than one monomer constituent), which may be either linear or branched. If a heteropolymer is derived from two monomer constituents, the polyolefin may assume any copolymer chain arrangement, including those of a block copolymer or a random copolymer. Similarly, if the polyolefin is a heteropolymer derived from more than two monomer constituents, it may likewise be a block copolymer or a random copolymer. In certain aspects, the polyolefin may be polyethylene (PE), polypropylene (PP), or a blend of polyethylene (PE) and polypropylene (PP), or multi-layered structured porous films of PE and/or PP. For example, commercially available polyolefin porous separator membranes include CELGARD® 2500 (a monolayer polypropylene separator) and CELGARD® 2320 (a trilayer polypropylene/polyethylene/polypropylene separator) available from Celgard LLC.

When the separator 26 is a microporous polymeric separator, it may be a single layer or a multi-layer laminate, which may be fabricated from either a dry or a wet process. For example, in certain instances, a single layer of the polyolefin may form the entire separator 26. In other aspects, the separator 26 may be a fibrous membrane having an abundance of pores extending between the opposing surfaces and may have an average thickness of less than a millimeter, for example. As another example, however, multiple discrete layers of similar or dissimilar polyolefins may be assembled to form the separator 26. The separator 26 may also comprise other polymers in addition to the polyolefin such as, but not limited to, polyethylene terephthalate (PET), polyvinylidene fluoride (PVdF), a polyamide, polyimide, poly(amide-imide) copolymer, polyetherimide, and/or cellulose, or any other material suitable for creating the required porous structure. The polyolefin layer, and any other optional polymer layers, may further be included in the separator 26 as a fibrous layer to help provide the separator 26 with appropriate structural and porosity characteristics.

In certain aspects, the separator 26 may further include one or more of a ceramic material and a heat-resistant material. For example, the separator 26 may also be admixed with the ceramic material and/or the heat-resistant material, or one or more surfaces of the separator 26 may be coated with the ceramic material and/or the heat-resistant material. In certain variations, the ceramic material and/or the heat-resistant material may be disposed on one or more sides of the separator 26. The ceramic material may be selected from the group consisting of: alumina (Al₂O₃), silica (SiO₂), and combinations thereof. The heat-resistant material may be selected from the group consisting of: Nomex, Aramid, and combinations thereof.

Various conventionally available polymers and commercial products for forming the separator 26 are contemplated, as well as the many manufacturing methods that may be employed to produce such a microporous polymer separator 26. In each instance, the separator 26 may have an average thickness greater than or equal to about 1 micrometer or micron (μm) to less than or equal to about 50 μm, and in certain instances, optionally greater than or equal to about 1 μm to less than or equal to about 20 μm.

In various aspects, the separator 26 may be replaced with a solid-state electrolyte (“SSE”) and/or semi-solid-state electrolyte (e.g., gel) that functions as a separator. For example, the solid-state electrolyte and/or semi-solid-state electrolyte may be disposed between the positive electrode 24 and negative electrode 22. The solid-state electrolyte and/or semi-solid-state electrolyte facilitates transfer of lithium ions, while mechanically separating and providing electrical insulation between the negative and positive electrodes 22, 24. By way of non-limiting example, the solid-state electrolyte and/or semi-solid-state electrolyte may include a plurality of fillers, such as LiTi₂(PO₄)₃, LiGe₂(PO₄)₃, Li₇La₃Zr₂O₁₂, Li₃xLa_{2/3}-xTiO₃, Li₃PO₄, Li₃N, Li₄GeS₄, Li₁₀GeP₂S₁₂, Li₂S—P₂S₅, Li₆PS₅Cl, Li₆PS₅Br, Li₆PS₅I, Li₃OCl, Li_{2.99} Ba_{0.005}ClO, or combinations thereof. The semi-solid-state electrolyte may include a polymer host and a liquid electrolyte. The polymer host may include, for example, polyvinylidene fluoride (PVDF), polyvinylidene fluoride-hexafluoropropylene (PVDF-HFP), polyethylene oxide (PEO), polypropylene oxide (PPO), polyacrylonitrile (PAN), polymethacrylonitrile (PMAN), polymethyl methacrylate (PMMA), carboxymethyl cellulose (CMC), poly(vinyl alcohol) (PVA), polyvinylpyrrolidone (PVP), and combinations thereof.

The negative electrode 22 may include a second (or negative) current collector 32 and first and second negative electroactive material layers 50, 52 disposed on opposing sides of the negative current collector 32. The negative current collector 32 may be a metal foil, metal grid or screen, or expanded metal. The negative current collector may comprise copper (Cu), nickel (Ni), iron (Fe), or any other appropriate electrical conductive material known to those of skill in the art. The first negative electroactive material layer 50 may be the same as or different form the second negative electroactive material 52. The first and second negative electroactive material layers 50, 52 may include negative electroactive materials, for example, lithium host materials, that are capable of functioning as a negative terminal of a lithium-ion battery. The negative electroactive material layers 50, 52 can be defined by a plurality of negative electroactive material particles (not shown). Such negative electroactive material particles may be disposed in one or more layers so as to define the three-dimensional structure of the negative electroactive material layers 50, 52.

In various aspects, negative electroactive material layers 50, 52 may include carbonaceous negative electroactive materials (such as, graphite, hard carbon, soft carbon, and the like) and/or metallic negative electroactive materials (such as tin, aluminum, magnesium, germanium, and alloys thereof, and the like). In other variations, the negative electroactive material layers 50, 52 may include a silicon-based negative electroactive material. In further variations, the negative electroactive material layers 50, 52 may be composite layers including a combination of negative electroactive materials. For example, one or both of the first and second negative electroactive material layers 50, 52 may include a first negative electroactive material and a second electroactive material. A ratio of the first negative electroactive material to the second negative electroactive material may be greater than or equal to about 5:95 to less than or equal to about 95:5. In certain variations, the first and second negative electroactive materials may be independently selected from one or more carbonaceous materials, one or more metallic materials, one or more silicon-based electroactive materials, or combinations thereof.

In each variation, the negative electroactive material may be optionally intermingled with an electronically conductive material (i.e., conductive additive) that provide an electron conductive path and/or a polymeric binder material that improve the structural integrity of the negative electrode 22. For example, each negative electroactive material layers 50, 52 may include greater than or equal to about 70 wt. % to less than or equal to about 98 wt. %, and in certain aspects, optionally greater than or equal to about 80 wt. % to less than or equal to about 95 wt. %, of the negative electroactive material; greater than or equal to 0 wt. % to less than or equal to about 30 wt. %, and in certain aspects, optionally greater than or equal to about 0.5 wt. % to less than or equal to about 10 wt. %, of the electronically conducting material; and greater than or equal to 0 wt. % to less than or equal to about 20 wt. %, and in certain aspects, optionally greater than or equal to about 0.5 wt. % to less than or equal to about 10 wt. %, of the polymeric binder. The conductive material and/or binder as included in the negative electroactive material layers 50, 52 may be the same as or different form the conductive material and/or binder as included in the positive electroactive material layers 40, 42.

As illustrated, a non-reactive lithium mimic material 80 may be disposed between layers of the negative electrode 22. For example, in certain variations, the non-reactive lithium mimic material 80 may overlap with (or overlay) a first surface 60 of the negative electrode current collector 32 and also a first surface 62 of the first negative electroactive material layer 50. The first surface 60 of the negative electrode current collector 32 may face towards the positive electrode 24, while the first surface 62 of the first negative electrode material layer 50 may face away from the positive electrode 24. Although illustrated as a discontinuous layer, it should be recognized that the non-reactive lithium mimic material 80 can take a variety of configurations, including a continuous layer or one or more discontinuous pieces having a variety of shapes and sizes. The non-reactive lithium mimic material 80 may have a variety of thicknesses and/or porosities. Similarly, although not illustrated, in certain variations, one or more contaminants may exist (i.e., be disposed) on one or more exposed surfaces of the non-reactive lithium mimic material 80. Similarly, the non-reactive lithium mimic material film 80 may have a selected porosity and/or one or more areas of delamination or disbonding with the adjacent layers (i.e., negative electrode current collector 32) may be selected in each instance so as to permit evaluation of the ability of nondestructive evaluation (NDE) tests and methods to evaluate the same.

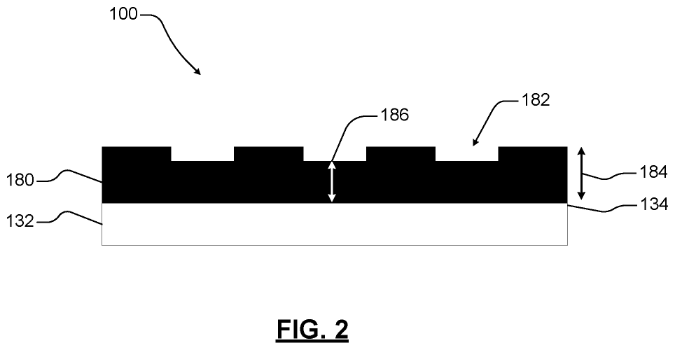

In various aspects, as illustrated in FIG. 2, a non-reactive lithium mimic material film 180 may be disposed on or near one or more surfaces of a current collector 132 enabling analysis of certain physical properties, or more specifically, certain defects, of the non-reactive lithium mimic material film 180. As illustrated, in certain variations, the non-reactive lithium mimic material film 180 may be disposed on a first surface 134 of the current collector 132. The current collector 132 may be a negative current collector like the negative current collector 32 illustrated in FIG. 1. The non-reactive lithium mimic material film 180 together with the current collector 132 may define an electrode assembly 100.

The non-reactive lithium mimic material film 180 may be disposed to form a substantially continuous layer that covers, for example, greater than or equal to about 95% of a total surface area of the surface 134 of the current collector 132 and has one or more areas or regions 182 of variable thicknesses allowing for the evaluation of the ability of nondestructive evaluation (NDE) tests and methods to evaluate coating uniformity and overall thicknesses. For example, in certain variations, the non-reactive lithium mimic material film 180 may have a first or overall thickness 184, while the one or more areas 182 have a second thickness 186 that is less than the first thickness 184. In certain variations, the first thickness 184 may be greater than or equal to about 2 micrometers (μm) to less than or equal to about 25 μm, and in certain aspects, optionally greater than or equal to about 15 μm to less than or equal to about 20 μm, while the second thickness 186 is greater than or equal to about 15 μm to less than or equal to about 100 μm, and in certain aspects, optionally greater than or equal to about 30 μm to less than or equal to about 40 μm. Although not illustrated, in certain variations, one or more contaminants may exist (i.e., be disposed) on one or more exposed surfaces of the non-reactive lithium mimic material film 180. Similarly, the non-reactive lithium mimic material film 180 may have a selected porosity and/or one or more areas of delamination or disbonding with the current collector 132 selected in each instance of evaluation of the ability of nondestructive evaluation (NDE) tests and methods to evaluate the same.

In each variation, the nondestructive testing may be implemented to detect regions of variations in thickness (or contaminants or porosities or delamination) of the non-reactive lithium mimic material 80 and/or non-reactive lithium mimic material film 180. In other variations, the non-reactive lithium mimic material 80 (as illustrated in FIG. 1) and/or non-reactive lithium mimic material film 180 (as illustrated in FIG. 2) may have similar electrical conductivity as lithium metal and eddy current nondestructive testing may be implemented in ambient conditions (e.g., temperature greater than or equal to about 20° C. to less than or equal to about 25° C. with relative humidity levels greater than or equal to about 20% to less than or equal to about 80%, an ambient water content of about 25,000 ppm, and 1 bar pressure) to identify and/or characterize variations in thickness and/or porosity of the non-reactive lithium mimic material 80 and/or non-reactive lithium mimic material film 180, as well as to identify and/or characterize delamination or disbonding between the non-reactive lithium mimic material 80 and/or non-reactive lithium mimic material film 180 and adjacent layers (i.e., negative electrode current collector 32 current collector 132, respectively). For example, eddy current nondestructive testing may include placing an eddy current probe at or near a surface of the prepared sample (e.g., non-reactive lithium mimic material 80 and/or non-reactive lithium mimic material film 180) and generating a magnetic field, for example, by passing alternating current (AC) through a coil, while an opposing current, known as the eddy current, is generated in the prepared sample through induction. The opposing eddy current has an associated magnetic field that can be measured by the eddy current probe. The presence of sample discontinuities may result in measurable disruptions of the associated magnetic field. Lithium has a room temperature electrical conductivity of about 11×10⁶ S/m and mimic materials having similar room temperature electrical conductivities include, for example, indium having an electrical conductivity of about 12×10⁶ S/m, tin having an electrical conductivity of about 9×10⁶ S/m, nickel having an electrical conductivity of about 14×10⁶ S/m, iron having an electrical conductivity of about 10×10⁶ S/m, and/or chromium having an electrical conductivity of about 8×10⁶ S/m.

In further variations, the non-reactive lithium mimic material 80 and/or non-reactive lithium mimic material film 180 may have similar thermal conductivity as lithium metal and flash thermography may be implemented in ambient conditions to identify and/or characterize variations in thickness and/or porosity of the non-reactive lithium mimic material 80 and/or non-reactive lithium mimic material film 180, as well as to identify and/or characterize delamination or disbonding between the non-reactive lithium mimic material 80 and/or non-reactive lithium mimic material film 180 and adjacent layers (i.e., negative electrode current collector 32 current collector 132, respectively) and surface contamination on exposed surfaces of the non-reactive lithium mimic material 80 and/or non-reactive lithium mimic material film 180. Flash thermography is a form of active thermography that involves imparting heat onto a prepared sample using electromagnetic radiation (e.g., light) and measuring the infrared response of the sample as it equilibrates at room temperature. Notably, delamination and disbonding impedes thermal flow. Lithium has a room temperature thermal conductivity of about 85 W/(m·K) and mimic materials having similar room temperature thermal conductivities include, for example, indium having a thermal conductivity of about 86 W/(m·K), tin having a thermal conductivity of about 67 W/(m·K), nickel having a thermal conductivity of about 92 W/(m·K), iron having a thermal conductivity of about 80 W/(m·K), and/or chromium having a thermal conductivity of about 90 W/(m·K).

In still other variations, the non-reactive lithium mimic material 80 and/or non-reactive lithium mimic material film 180 may have similar acoustic properties as lithium metal and air-coupled ultrasound may be implemented in ambient conditions to identify and/or characterize variations in thickness and/or porosity of the non-reactive lithium mimic material 80 and/or non-reactive lithium mimic material film 180, as well as to identify and/or characterize delamination or disbonding between the non-reactive lithium mimic material film 180 and the current collector 132 and surface contamination on exposed surfaces of the lithium mimic material film 180.

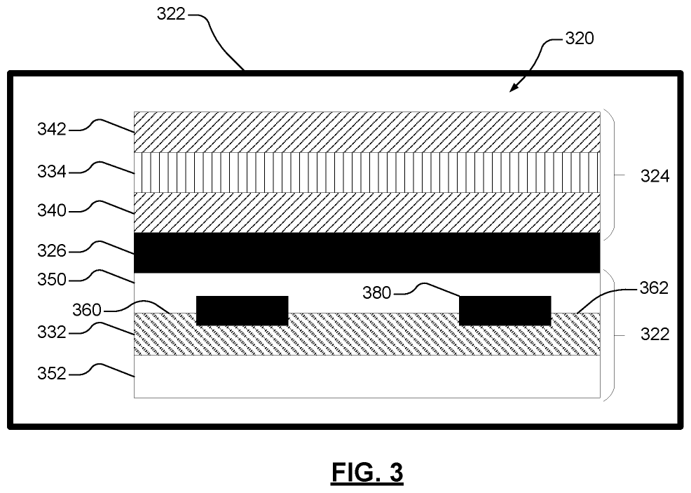

In various aspects, the present disclosure provides methods for safely handling lithium-containing samples for nondestructive evaluation (NDE) testing. For example, in certain variations, as illustrated in FIG. 3, an electrochemical cell 320 may be disposed (e.g., vacuum sealed) within a non-electrically conductive pouch material 322 so as to improve handing of the assembly in ambient conditions. Lithium-containing samples enclosed by the non-conductive pouch material 322 can be safely handled in ambient conditions. Like the electrochemical cell 20 illustrated in FIG. 1, the electrochemical cell 320 illustrated in FIG. 3 may be a dry cell with no voltage generation that includes a first (or positive) electrode 324 physically separated from a second (or negative) electrode 322 by a separating layer 326. Similarly, the positive electrode 324 may include a first (or positive) current collector 334 and first and second positive electroactive material layers 340, 342 disposed on opposing sides of the positive current collector 334; and the negative electrode 322 may include a second (or negative) current collector 332 and first and second negative electroactive material layers 350, 352 disposed on opposing sides of the negative current collector 332.

Unlike the electrochemical cell 20, however, the electrochemical cell 320 may include a lithium-containing material 380 disposed between layers of the negative electrode 322. For example, in certain variations, the lithium-containing material 380 may overlap with (or overlay) a first surface 360 of the negative electrode current collector 332 and also a first surface 362 of the first negative electroactive material layer 350. The lithium-containing material 380 may make contact with the first surface 360 of the negative electrode current collector 332 and also the first surface 362 of the first negative electroactive material layer 350. Similarly, although the lithium-containing material 380 is illustrated as a discontinuous layer, it should be recognized that the lithium-containing material 380 can take a variety of configurations, including a continuous layer or one or more discontinuous pieces having a variety of shapes and sizes. For example, in certain variations, lithium-containing material 380 may be co-extensive with the first surface 360 of the negative electrode current collector 332 and/or the first surface 362 of the first negative electroactive material layer 350. Further still, in certain variations, the lithium-containing material 380 may define one or both of the negative electroactive material layers 350, 352. In each instance, the lithium-containing material 380 may have a variety of thicknesses and/or porosities. Similarly, although not illustrated, in certain variations, one or more contaminants may exist (i.e., be disposed) on one or more exposed surfaces of the lithium-containing material 380. Similarly, the lithium-containing material 380 may have a selected porosity and/or one or more areas of delamination or disbonding with the adjacent layers (i.e., negative electrode current collector 332) may be selected in each instance so as to permit evaluation of the ability of nondestructive evaluation (NDE) tests and methods to evaluate the same. Although a lithium-containing material 380 is discussed in the context of FIG. 3, it should be recognized that in certain instances a lithium mimic material as detailed above may replace the lithium-containing material 380.

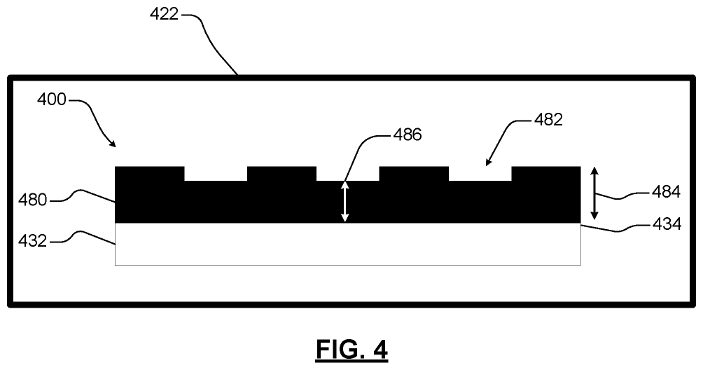

In various aspects, the present disclosure provides methods for safely handling lithium-containing samples for nondestructive evaluation (NDE) testing. For example, in certain variations, as illustrated in FIG. 4, an electrode assembly 400 may be disposed (e.g., vacuum sealed) within a non-electrically conductive pouch material 422 so as to improve handing of the assembly in ambient conditions. Lithium-containing samples enclosed by the non-conductive pouch material 422 can be safely handled in ambient conditions. The electrode assembly 400 includes a lithium-containing material 480 disposed on or near one or more surfaces of a current collector 432. Like the electrode assembly 100 illustrated in FIG. 2, the electrode assembly 400 illustrated in FIG. 4 may be configured to enable analysis of certain physical properties, or more specifically, certain defects, of the lithium-containing material 480. For example, the lithium-containing material 480 may be disposed to form a substantially continuous layer that covers, for example, greater than or equal to about 95% of a total surface area of the current collector 432 and has one or more areas or regions 482 of variable thicknesses allowing for the evaluation of the ability of nondestructive evaluation (NDE) tests and methods to evaluate coating uniformity and overall thicknesses. For example, in certain variations, the lithium-containing material 480 may have a first or overall thickness 484, while the one or more areas 482 have a second thickness 486 that is less than the first thickness 484. In certain variations, the first thickness 484 may be greater than or equal to about 2 micrometers (μm) to less than or equal to about 25 μm, and in certain aspects, optionally greater than or equal to about 15 μm to less than or equal to about 20 μm, while the second thickness 486 is greater than or equal to about 15 μm to less than or equal to about 100 μm, and in certain aspects, optionally greater than or equal to about 30 μm to less than or equal to about 40 μm. Although not illustrated, in certain variations, one or more contaminants may exist (i.e., be disposed) on one or more exposed surfaces of the lithium-containing material 480. Similarly, the lithium-containing material 480 may have a selected porosity and/or one or more areas of delamination or disbonding with the current collector 432 selected in each instance of evaluation of the ability of nondestructive evaluation (NDE) tests and methods to evaluate the same. Although a lithium-containing material 480 is discussed in the context of FIG. 4, it should be recognized that in certain instance a lithium mimic as detailed above may replace the lithium-containing material 480

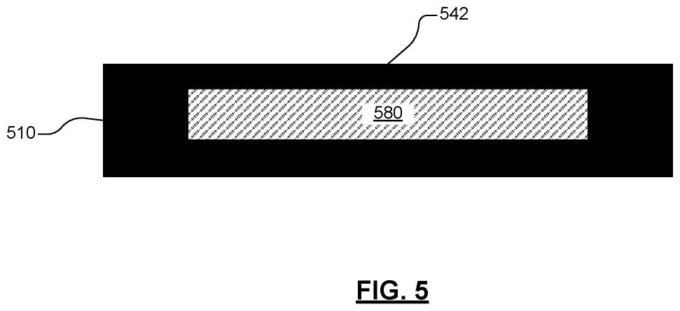

In various aspects, the present disclosure provides methods for safely handling lithium-containing samples for nondestructive evaluation (NDE) testing. For example, in certain variations, as illustrated in FIG. 5, a lithium-containing material 580 may be embedded withing (e.g., disposed between parallel layers of and surround by) a substrate 510, such as a copper mesh or a copper expanded metal foil, so as to improve handling of the assembly in ambient conditions. It will be understood that, in certain variations, the substrate 510 may be a current collector. For example, as illustrated, in certain variations, the substrate 510 may include a first layer and a second layer and the lithium-containing material 580 may be disposed between the first layer and the second layer. In certain aspects, the first and second copper layers may join together at terminal ends around the lithium-containing material 580 to completely encase the lithium-containing material 580. Although a lithium-containing material 580 is discussed in the context of FIG. 5, it should be recognized that in certain instance a lithium mimic material as detailed above may replace the lithium-containing material 580.

In various aspects, the present disclosure provides methods for preparing electrode assemblies and electrochemical cells. For example, the present disclosure provides example methods for preparing electrode assemblies including a non-reactive lithium mimic material disposed on or near (e.g., with one or more inventing layers or materials) one or more surfaces of a current collector, like the electrode assembly 100 illustrated in FIG. 2. In certain variations, an additive manufacturing process (or three-dimensional printing) may be used where the non-reactive lithium mimic material is disposed on or near the current collector in one or more layers, for example, by spreading a thin layer of powder and melting the powder and the layer beneath using a laser or electron beam to form a solid material. In other variations, a non-reactive lithium mimic material in powder or shavings form may be disposed on or near the current collector. Shavings may be slices having a greatest thickness less than or equal to the thickness of the underlying current collector. In still other variations, a lamination process, for example a roll-to-roll process, may be used to dispose the non-reactive lithium mimic material film on or near the current collector. In still other variations, sputter and/or spray processes, for example a vapor deposition process such as vacuum thermal evaporation and/or pulsed laser deposition, may be used to dispose the non-reactive lithium mimic material film on or near the current collector. Further still, in certain variations, the lithium mimic material may be applied as a metallic paint layer where metallic particles are suspended in a carrier solution and evaporated after application of the paint.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An electrode assembly for nondestructive testing in ambient conditions, the electrode assembly comprising:
a current collector; and
a non-reactive lithium mimic material disposed on or near one or more surfaces of the current collector or embedded in the current collector.

2. The electrode assembly of claim 1, wherein the non-reactive lithium mimic material has an electrical conductivity that is within about 10% of an electrical conductivity of lithium.

3. The electrode assembly of claim 1, wherein the non-reactive lithium mimic material has an electrical conductivity greater than or equal to about 7×10⁶ S/m to less than or equal to about 15×10⁶ S/m.

4. The electrode assembly of claim 1, wherein the non-reactive lithium mimic material has a thermal conductivity that is within about 10% of a thermal conductivity of lithium.

5. The electrode assembly of claim 1, wherein the non-reactive lithium mimic material has a thermal conductivity greater than or equal to about 60 W/(m·K) to less than or equal to about 100 W/(m·K).

6. The electrode assembly of claim 1, wherein the non-reactive lithium mimic material is selected from the group consisting of: indium, tin, iron, nickel, chromium, and alloys thereof.

7. The electrode assembly of claim 1, wherein the non-reactive lithium mimic material defines a continuous layer having one or more first regions with a first thickness and one or more second regions with a second thickness, wherein the first thickness is different from the second thickness.

8. The electrode assembly of claim 1, wherein the non-reactive lithium mimic material is disposed on a first surface of the current collector and an interface defined between the non-reactive lithium mimic material and the current collector comprises one or more selected regions of delamination.

9. The electrode assembly of claim 1, wherein the non-reactive lithium mimic material defines a discontinuous layer over the one or more surface of the current collector.

10. The electrode assembly of claim 1, further comprising:
a continuous electroactive material layer having a major dimension parallel with a major dimension of the current collector, the non-reactive lithium mimic material overlapping a portion of the non-reactive lithium mimic and a portion of the electroactive material layer at an interface of the electroactive material layer and the current collector.

11. The electrode assembly of claim 1, wherein the non-reactive lithium mimic material is disposed on or near the one or more surfaces of the current collector and the electrode assembly is sealed in a non-conductive pouch.

12. The electrode assembly of claim 1, wherein the non-reactive lithium mimic material is embedded in the current collector, and the current collector comprises a copper mesh or a copper expanded metal foil.

13. An electrode assembly for nondestructive testing in ambient conditions, the electrode assembly comprising:
a current collector;
a lithium metal material or non-reactive lithium mimic material disposed on or near one or more surfaces of the current collector or embedded in the current collector to form an electrode assembly; and
a non-conductive pouch holding the electrode assembly.

14. The electrode assembly of claim 13, wherein the lithium metal material or non-reactive lithium mimic material defines a continuous layer having one or more first regions with a first thickness and one or more second regions with a second thickness, wherein the first thickness is different from the second thickness.

15. The electrode assembly of claim 13, wherein the lithium metal material or non-reactive lithium mimic material defines a discontinuous layer over the one or more surfaces of the current collector.

16. The electrode assembly of claim 13, wherein the lithium metal material or non-reactive lithium mimic material is embedded in the current collector, and the current collector comprises a copper mesh or a copper expanded metal foil.

17. The electrode assembly of claim 13, further comprising:
a continuous electroactive material layer having a major dimension parallel with a major dimension of the current collector, the non-reactive lithium mimic material overlapping a portion of the non-reactive lithium mimic and a portion of the electroactive material layer at an interface of the electroactive material layer and the current collector.

18. The electrode assembly of claim 13, wherein the non-reactive lithium mimic material has an electrical conductivity greater than or equal to about 7×10⁶ S/m to less than or equal to about 15×10⁶ S/m.

19. The electrode assembly of claim 13, wherein the non-reactive lithium mimic material has a thermal conductivity greater than or equal to about 60 W/(m·K) to less than or equal to about 100 W/(m·K).

20. The electrode assembly of claim 13, wherein the non-reactive lithium mimic material is selected from the group consisting of: indium, tin, iron, nickel, chromium, alloys, and combinations thereof.
